(19) United States
(12) Patent Application Publication
(10) Pub. No.: US 2008/0319386 A1
(43) Pub. Date: Dec. 25, 2008

(54) Forwardly directable fluid jet crossing catheter

(75) Inventors: Michael J. Bonnette, Minneapolis, MN (US); Eric J. Thor, Arden Hills, MN (US); David B. Morris, Anoka, MN (US); Debra M. Kozak, Forest Lake, MN (US)

Correspondence Address: GREGORY L BRADLEY;MEDRAD INC, ONE MEDRAD DRIVE, INDIANOLA, PA, 15051, US

(73) Assignee: Possis Medical, Inc.

(21) Appl. No.: 11/891,450

(22) Filed: Aug. 10, 2007

Related U.S. Application Data
(60) Provisional application No. 60/936,507, filed on Jun. 20, 2007.

(51) Int. Cl. A61M 25/085

(52) U.S. Cl. 604/95.02; 604/164.13; 604/529

(57) ABSTRACT

A forwardly directable fluid jet crossing catheter having a positionable jet body which can be longitudinally and rotationally positioned in order to direct a fluid jet stream to impinge, break through, and cross a chronic total occlusion. The distal end of the catheter includes a tip of super-elastic material having a flexible bend aligned within the distal portion of a sheath which tip is directable in response to the longitudinal and rotational orientation of a positionable jet body, whereby a fluid jet stream can be controlled in angular and rotational directions.

### CROSS REFERENCES TO RELATED APPLICATIONS

This application claims priority from the earlier filed U.S. Provisional Application No. 60/936,507 filed Jun. 20, 2007, entitled “Forwardly Directable Fluid Jet Crossing Catheter”, and is hereby incorporated into this application by reference as if fully set forth herein.

### BACKGROUND OF THE INVENTION

1. Field of the Invention

The present invention relates to the field of catheters, and more particularly, relates to a forwardly directable fluid jet crossing catheter used for the purpose of crossing a Chronic Total Occlusion (CTO), whereby a moderate speed and safe velocity fluid jet is used to wear away arterial lesions forming a CTO and advancing therethrough. Chronic total occlusions are arterial lesions that have progressed to the point where there is no flow through the vessel (total occlusion). Furthermore, it is generally considered highly difficult to cross a CTO with a standard support guidewire. In other words, if a total occlusion is easily crossed with a standard guidewire, it is not a chronic total occlusion. Furthermore, coronary chronic total occlusions have been characterized as having tough fibrous and even calcific caps with a softer interior. This invention is intended to help a guidewire penetrate the chronic total occlusion by directing a moderate speed fluid jet at the occlusion.

The forwardly directable fluid jet crossing catheter is designed to cross chronic total occlusions in a peripheral or coronary artery. Bodies of scientific evidence have indicated that after opening by crossing a coronary chronic total occlusion in a patient, the patient is benefited thereby. Although the presence of a chronic total occlusion usually means there is some collateralization, opening of a chronic total occlusion provides a greater flow reserve. As a result, the opening of chronic total occlusions in a patient has been shown to have improved patient morbidity and mortality. Furthermore, a peripheral procedure can be expedited by crossing peripheral chronic total occlusions. In the case of critical limb ischemia cases, the slow progression of a peripheral artery disease may result in total occlusions in peripheral arteries that are difficult to cross with conventional wires. Other methods, such as the use of a laser, can facilitate this crossing capability for peripheral arteries depending on the amount of calcification.

2. Description of the Prior Art

The crossing of chronic total occlusions is a relatively new treatment modality. As such, the field is not mature with products that are proven in this challenging task, especially for chronic total occlusions in a coronary artery. There are a few products that are being used for this coronary treatment. In general, the first choice of physicians is the use of improved guidewires of which there are many. The Confienza Conquest wire is an example of a very stiff tip wire used to penetrate the fibrous cap of a chronic total occlusion. However, the use of this type of stiff wires for chronic total occlusions is challenging and time consuming resulting in an increased radiation exposure to the patient. Other devices that have been tried include the FrontRunner by Lumend which is a clamshell type device for mechanically opening its blunt jaws at the face of the chronic total occlusion. This device was unsuccessful all the time so it was not seen as being reliable. Another device is the Safe Cross system from Interluminal Therapeutics. This system consists of a radio frequency ablation wire coupled with an Optical Coherence Detection device to ensure that the wire does not burn through the vessel wall. Although this system is considered generally reliable by trained professionals, it has some limitations. First, the method is slow. Second, if a channel is burned next to the vessel wall, it can be difficult to direct the wire to take an alternative path. Another device is a re-entry device by Lumend, (Outback Catheter). This device provides a procedure for crossing a peripheral chronic total occlusion by purposely directing a wire into the subintimal space of the vessel. Once the wire is beyond the chronic total occlusion, the re-entry catheter directs the wire back into the true lumen. Although this is a novel technique, it is not uniformly accepted, nor comfortable for physicians to perform this type of procedure. Lasers can be used to cross peripheral chronic total occlusions and can facilitate the crossing capability for peripheral arteries depending on the amount of calcification.

### SUMMARY OF THE INVENTION

The general purpose of the present invention is to provide a forwardly directable fluid jet crossing catheter. The forwardly directable fluid jet crossing catheter uses a single directable saline fluid jet to penetrate through a chronic total occlusion. The concept for the present invention stemmed from vessel safety testing that had been done with thrombectomy catheters. Collective findings of numerous animal studies have shown that the side exhaust flow velocities from thrombectomy catheters were safe although the internal fluid jets were so fast that they could damage an artery when it was contacted. Thrombectomy catheters, using cross stream technology, have two distinct sets of windows (or orifices). There is one set of inflow windows that is near the origin of the internal high velocity fluid jets and another set of side exhaust windows that is located proximally. The velocity of the flow leaving the side exhaust windows, i.e., cross stream jets, for various thrombectomy catheters varies between 5 m/s and 15 m/s. Vessel safety testing has shown that vessel damage is associated with the inflow windows rather than the outflow windows. The internal high velocity of the fluid jet is on the order of 140-200 meters/second. Furthermore, testing has shown that thrombectomy catheters are highly efficacious on soft fresh clots. However, as a clot becomes more aged and organized, the strength of the clot can increase in orders of magnitude and the thrombectomy catheters become less efficacious, unless used in combination with fibrinolytics. Hence, the side exhaust velocity of 5-15 m/s is insufficient to penetrate this organized clot. Therefore, the concept behind the forwardly directable fluid jet crossing catheter, which can also be referred to as a front spray catheter, is to choose a velocity that is less than the dangerous high velocity fluid jets, i.e., less than 140 meters/second, such that it will still be safe for contacting the vessel wall, and yet higher than the side exhaust velocity, i.e., greater than 15 meters/second, so that it will be efficacious in penetrating through a tough organized clot.

A significant feature of the present invention is the small crossing profile and directability of the fluid jet. Since the device of this invention is used to cross chronic total occlusions, the smaller the crossing profile the better the chance to successfully navigate across the lesion. Therefore, an exhaust lumen becomes a feature that is not used in order to reduce the crossing profile. Secondly, the device has a directable tip. The main components of the forwardly directable fluid jet crossing catheter generally include: a flexible catheter tube having a flexible coated tube and connected flexible tubular polymer sheath; a smaller profile jet body having a proximally located flexible tube, preferably of stainless steel; a connected distally located flexible tube, preferably of nitinol; and an operating handle. Together, the combination of the flexible stainless steel tube and the flexible nitinol tube of the jet body functions substantially as a flexible hypotube for the delivery of a fluid, at a suitable pressure strength, to the directable fluid jet. The greater portion (i.e., the flexible high pressure tube) of the jet body aligns along the interior of the flexible catheter tube where one of its ends is directly and closely associated with the operating handle and where its other end (i.e., the flexible nitinol tube) extends along, within, and at various lengths from the distal end of the catheter tube sheath. The distally located nitinol tube includes a bend having a memory shape located a short distance from the distal tip of the nitinol tube. Nitinol can be shaped at elevated temperatures and, as such, portions of the nitinol tubing are heat treated with a memory shape or heat set. Thus, the nitinol tubing is flexible (Martensitic) so that it can be bent angularly, longitudinally and otherwise controlled with respect to the sheath by the control handle which is operated by a physician.

The distal portion of the nitinol tube can align in different relationships within the lumen of the sheath. When, by manipulation of components of the control handle, the nitinol tube is withdrawn into the sheath, the bend in the nitinol tube straightens out in order to provide a medium velocity fluid jet directed substantially along the axis of the sheath. Conversely, by manipulation of components of the control handle, if the tip of the nitinol tubing is extended outwardly from the sheath, the preset bend in the nitinol tube assumes a bent shape in order to provide a medium velocity fluid jet directed in a controlled angle direction with respect to the axis of the sheath. Secondly, in another relationship, the sheath can be torsionally directed and the bent nitinol tube can be torsionally directed, each independent of the other by manipulation of components of the control handle. By changing the relationship of the longitudinal or torsional positions between the nitinol tube and the proximal sheath, the device, as well as the direction of the fluid jet, can be steered or guided in a multitude of directions. This forms the principle for directing a suitable strength fluid jet out the front of the forwardly directable fluid jet crossing catheter. A dedicated guidewire lumen is affixed to the tip of the sheath and terminates at a guidewire tube exit region at the joint between the sheath and the flexible coated tube in order that a guidewire can be utilized by the forwardly directable fluid jet crossing catheter.

According to one embodiment of the present invention, there is provided a forwardly directable fluid jet crossing catheter having major components, including: a flexible coated tube; a flexible sheath; and an abbreviated flexible guidewire tube attached to and extending along the distal portion thereof constituting a flexible catheter tube; a stainless steel flexible high pressure tube having a coil near the proximal end thereof; a high pressure tube adapter; a ferrule; a high pressure connector and a flexible nitinol tube having a positionable tip with a memory shape or set attached to the distal end of the flexible stainless steel tube constituting the majority of a jet body; a configured tubular handle body; a positionable high pressure tube mount; a tubular actuator constituting an operating handle; and other components facilitating attachment of the flexible coated tube to the distal end of the operating handle. Another embodiment of the present invention foregoes the use of a coil in the flexible high pressure stainless steel tube and uses a control wire to influence the direction of the positionable tip at the distal portion of the flexible nitinol tube.

One significant aspect and feature of the forwardly directable fluid jet crossing catheter of the present invention is the ability to cross chronic total occlusions.

Another significant aspect and feature of the present invention is the use of a forwardly directed fluid jet in combination with a directable catheter tip.

Still another significant aspect and feature of the present invention is the use of a section of an annealed nitinol tubing, distal to a section of super-elastic nitinol tubing, for the purpose of forming a directable and shapeable atramatic tip.

Another significant aspect and feature of the present invention is the heat processing of the nitinol tubing to form a soft and flexible nitinol tube which provides a flexible and safe distal nitinol tube.

A further significant aspect and feature of the present invention is the use of a relatively stiffer sheath aligned over and about a pre-bent section of nitinol tube in a jet body for use in the angular directing of the catheter tip, and thus provide a fluid jet stream from the forwardly directable fluid jet crossing catheter.

Another significant aspect and feature of the present invention is the use of a forwardly directed fluid jet stream in combination with a directable catheter tip, whereby the forwardly directed fluid jet can be directed along the longitudinal axis or directed offset from the longitudinal axis of a surrounding sheath.

Another significant aspect and feature of the present invention is the use of an operating handle to control the positioning of a nitinol tube of a jet body linearly along the longitudinal axis of a surrounding sheath and/or rotationally about the longitudinal axis of a surrounding stiffer sheath, whereby a formed bend in the nitinol tube can be oriented in multiple positions with respect to the surrounding and stiffer sheath.

Another significant aspect and feature of the present invention is the use of a forwardly directed fluid jet stream emitting device in combination with a dedicated guidewire lumen.

Yet another significant aspect and feature of the present invention is a forwardly directed fluid jet having a jet velocity between 15 meters/second to 100 meters/second.

A still further significant aspect and feature of the present invention is providing volumetric flow rate for a forward directed fluid jet to be between 5 ml/min to 120 ml/min.

A still further significant aspect and feature of the present invention is a control wire retraction mechanism for purposes of directing the tip of the forwardly directable fluid jet crossing catheter.

Having thus briefly described embodiments of the present invention and having mentioned some significant aspects and features of the present invention, it is the principal object of the present invention to provide a forwardly directable fluid jet crossing catheter.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is an isometric view of a forwardly directable fluid jet crossing catheter of the present invention;

FIG. 2 is an expanded view of FIG. 1 showing a jet body removed from and exterior to a flexible catheter tube;

FIG. 3 is an exploded isometric view of the forwardly directable fluid jet crossing catheter along line 3-3 of FIG. 2;

FIG. 4 is a side view in cross section of the forwardly directable fluid jet crossing catheter showing components located proximal to the flexible coated tube;

FIG. 5 is an assembled view of the components of FIG. 4;

FIG. 6 is an isometric view of the guidewire tube exit region where the sheath is shown removed from and distant to the distal end of the flexible coated tube;

FIGS. 7a, 7b and 7c are cross section views along lines 7a-7a, 7b-7b and 7c-7c of FIG. 1 showing cross section views of the flexible catheter tube;

FIG. 8 is a partial cross section view showing the operating handle rotated in relation to the proximally located components of the jet body;

FIGS. 9a, 9b and 9c illustrate the longitudinal positional and slideable relationships of the tip, the bend and a distal portion of the nitinol tube of the jet body to the distal end of the sheath;

FIG. 10 shows the distal portion of the sheath and various rotational positions of the tip rotated therein;

FIG. 11 shows the distal end of the sheath and the tip of the forwardly directable fluid jet crossing catheter aligned in a vessel having a chronic total occlusion;

FIG. 12 is an exploded view similar to FIG. 4 showing the proximal portion of an alternative embodiment jet body;

FIG. 13 is similar to FIG. 5 showing an assembled view of the proximal portion of an alternative embodiment jet body and a control handle; and,

FIGS. 14a, 14b and 14c are similar to FIGS. 9a, 9b and 9c showing the attachment of the distal end of a control wire to a nitinol tube at a location just distal of a bend.

### DETAILED DESCRIPTION OF THE PREFERRED EMBODIMENTS

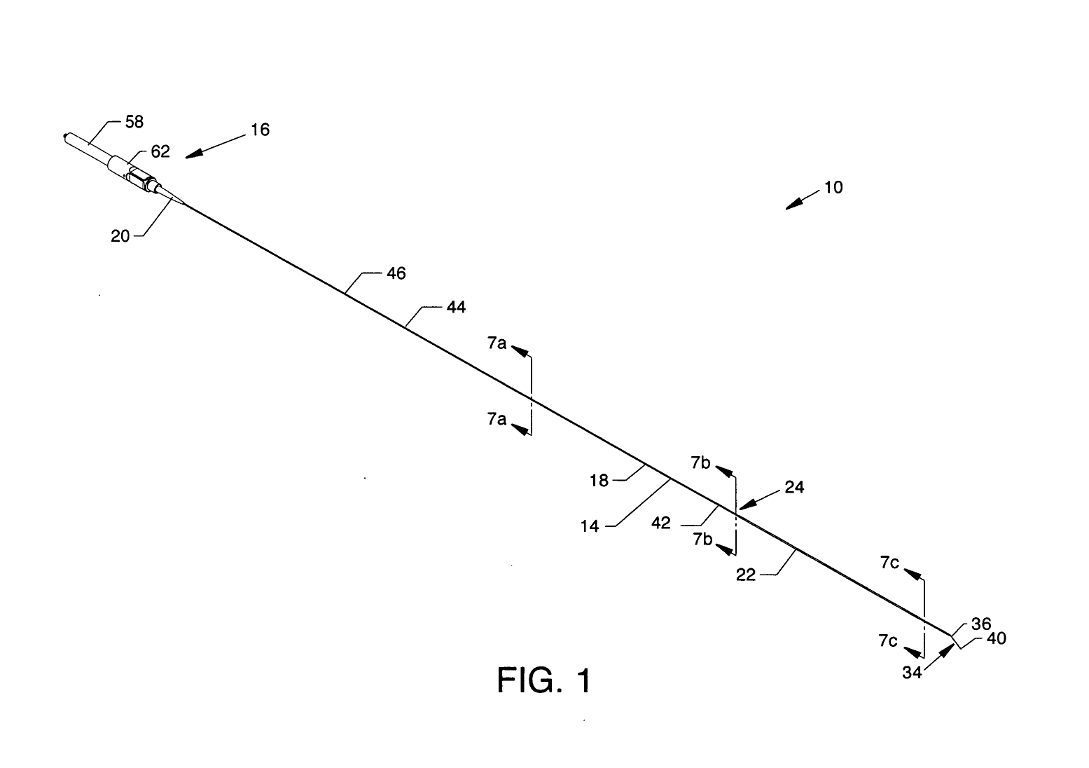

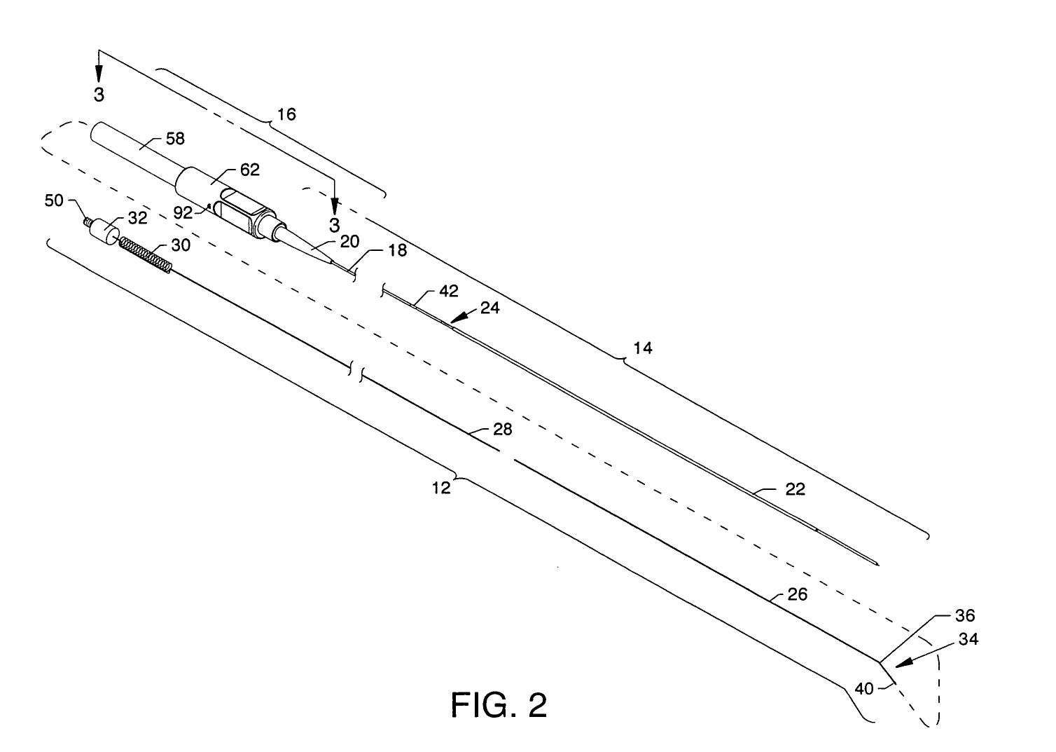

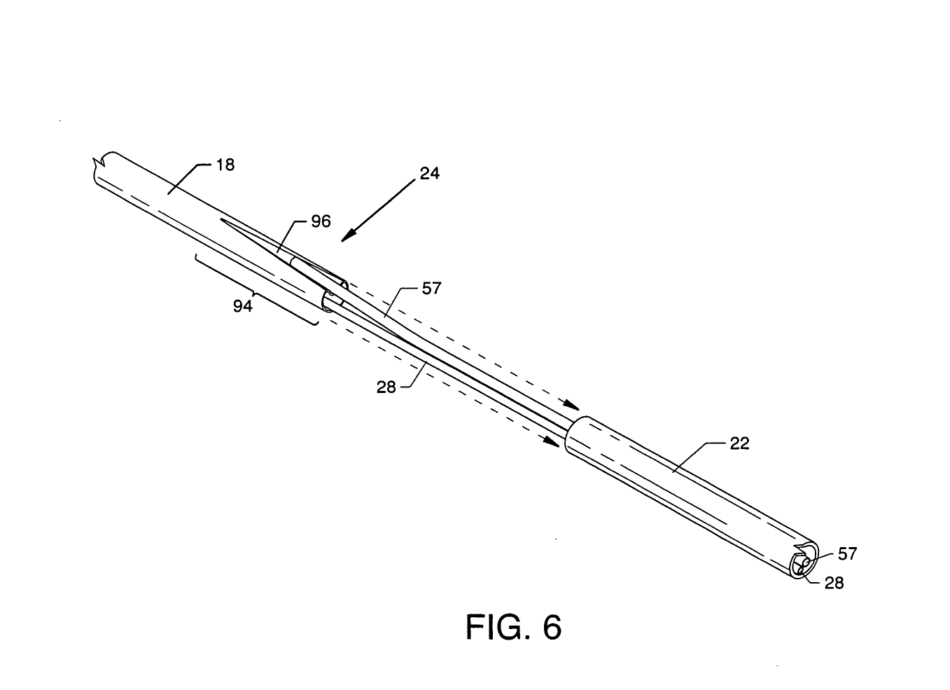

FIG. 1 is an isometric view of a forwardly directable fluid jet crossing catheter 10 of the present invention, and FIG. 2 is an expanded view of FIG. 1 showing a jet body 12 removed from and exterior to a flexible catheter tube 14. The proximal end of the flexible catheter tube 14 and the proximal end of the jet body 12 align and secure within an operating handle 16 located at the proximal end of the forwardly directable fluid jet crossing catheter 10. The flexible catheter tube 14 includes a flexible coated tube 18 extending distally from a flexible strain relief 20 located at one end of the operating handle 16 and also includes a sheath 22 being slightly larger in diameter than the flexible coated tube 18 where the proximal end of the sheath 22 is aligned over and about the smaller distal end of the flexible coated tube 18 at a guidewire tube exit region 24. The configuration of the guidewire tube exit region 24 is closely related to and described in patent application Ser. No. 11/096,592 filed Apr. 1, 2005, entitled “Rapid Exchange Fluid Jet Thrombectomy Catheter and Method”, which is pending. The guidewire tube exit region 24 is shown in detail in FIG. 6. The jet body 12 includes a distally located nitinol tube 26 connected to and extending distally from a proximally located flexible high pressure tube 28, preferably of stainless steel. A tubular coil 30 is continuous with proximal end of the flexible high pressure tube 28. The proximal end of the tubular coil 30 is suitably secured within a high pressure tube adapter 32, as later described in detail. The distal portion of the nitinol tube 26 includes a tip 34 which is annealed and atraumatic being distal to a super-elastic shaped bend 36. The tip 34 is soft, flexible and shapeable. The portion of the nitinol tube 26 proximal to the bend 6 is super-elastic, as is the bend 36, and each are soft and bendable and predisposed to return to an original memory shape. A radiopaque marker band 40 secures about the distal portion of the tip 34. Radiopaque marker bands are also located along and about the flexible catheter tube 14, and more specifically, along and about the flexible coated tube 18. A radiopaque marker band 42 is located about the flexible coated tube 18 proximal to the guidewire tube exit region 24. Radiopaque marker bands 44 and 46 are located about the flexible coated tube 18 at the proximal region of the flexible catheter tube 14.

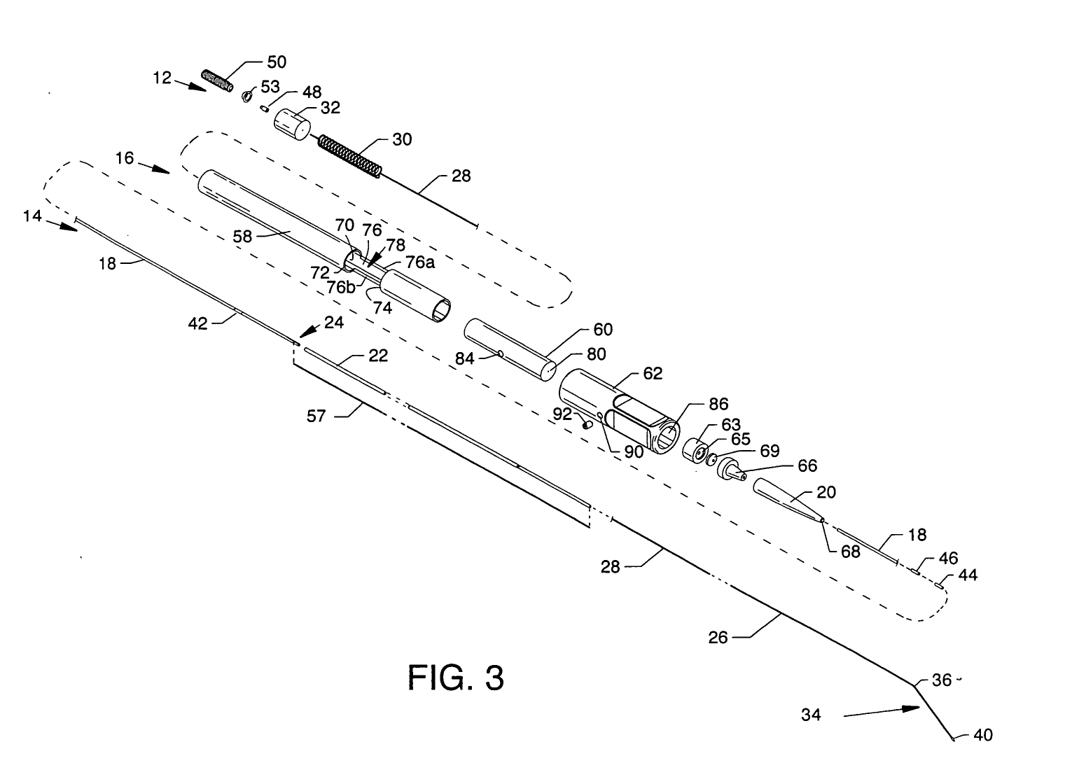

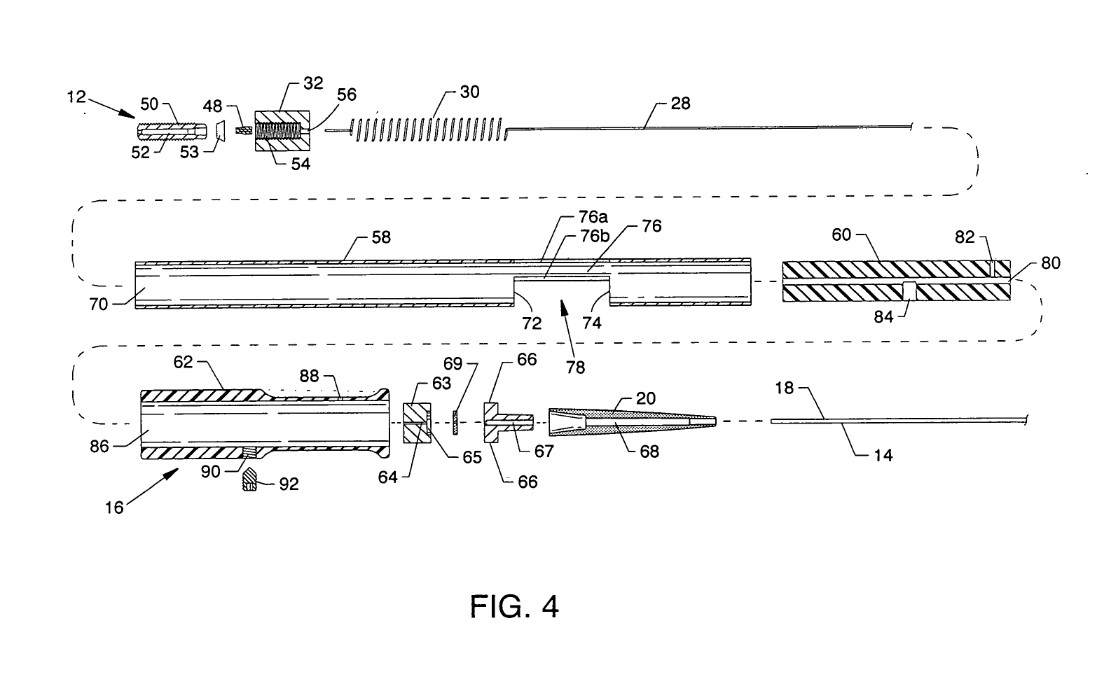

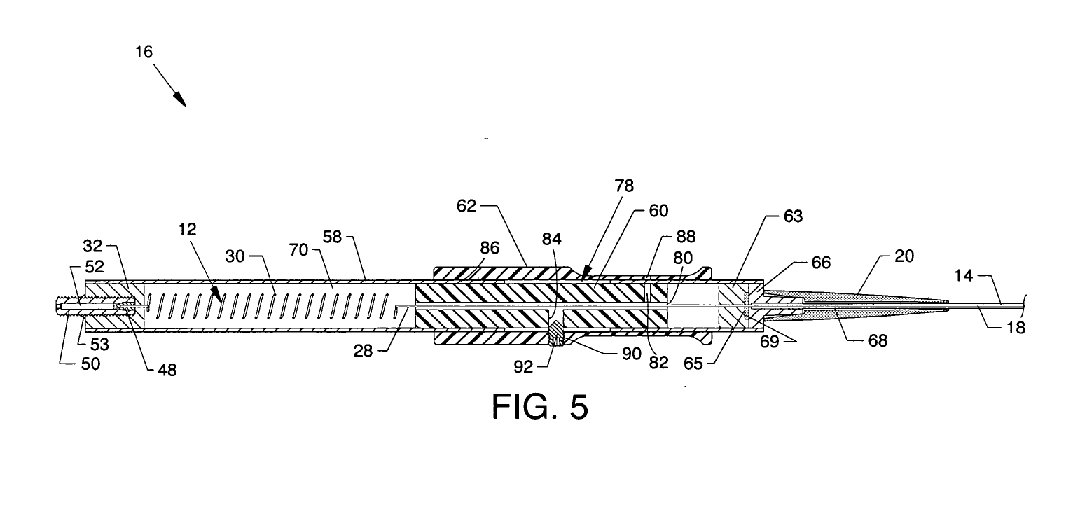

FIG. 3 is an exploded isometric view of the forwardly directable fluid jet crossing catheter 10, FIG. 4 is a side view in cross section along line 3-3 of FIG. 2 of the forwardly directable fluid jet crossing catheter 10 showing components located proximal to the flexible coated tube 18, and FIG. 5 is an assembled view of the components of FIG. 4. With reference to FIGS. 3, 4 and 5, description of components, component function, operational aspects and other attributes of the present invention included therein follow.

Other components of the jet body 12, in addition to the previously described components of the jet body 12, include a ferrule 48 for connection to the portion of the flexible high pressure tube 28 which is proximal to the tubular coil 30, and an externally threaded high pressure connector 50 having a bore 52 which receives the ferrule 48 and proximal end of the flexible high pressure tube 28. Adhesive 53 surrounds the outer junction of the high pressure connector 50 and the high pressure tube adapter 32. The cylindrically shaped high pressure tube adapter 32 includes a threaded hole 54 and a bore 56 which is continuous with and in communication with the threaded hole 54. The threaded hole 54 receives the high pressure connector 50 and the bore 56 allows passage of the proximal end of the flexible high pressure tube 28. A greater portion of the jet body 12, i.e., the portion of the flexible high pressure tube 28 extending through and beyond the strain relief 20, is located in the lumen of the flexible coated tube 18 of the flexible catheter tube 14 and extends further through the guidewire tube exit region 24 of the flexible catheter tube 14, and thence through the lumen of the sheath 22 of the flexible catheter tube 14 to emerge at the distal end of the sheath 22 where the shape, orientation and extension therethrough at such distal end can be influenced by the manipulation of the operating handle 16. A guidewire tube 57, the proximal end of which is located at and which is externally accessible at the guidewire tube exit region 24, extends distally from the guidewire tube exit region 24 along the lumen of the sheath 22 of the flexible catheter tube 14 to terminate at the distal end of the sheath 22. The guidewire tube 57 can be used in conjunction with over-the-wire guidewire usage, as referenced in patent application Ser. No. 11/096,592 filed Apr. 1, 2005, entitled “Rapid Exchange Fluid Jet Thrombectomy Catheter and Method”, which is pending.

The central portion of the operating handle 16 is comprised of interacting components which control the interaction of components of the jet body 12 to orient the bend 36 and tip 34 of the nitinol tube 26 with respect to the distal end of the sheath 22. A configured tubular handle body 58, preferably of stainless steel, serves as a mount for: a positionable high pressure tube mount 60, preferably of nylon; an ergonomic tubular actuator 62, preferably of Delrin®; a seal mount 63 preferably of stainless steel having a bore 64 and a connected annular recess 65; and a strain relief 66 mount, preferably of stainless steel, having a bore 67. Other closely associated components which are in a fixed location at the distal portion of the operating handle 16 include the flexible strain relief 20 having a configured bore 68 and a flexible seal 69, the latter of which is described in patent application Ser. No. 10/455,096 filed Jun. 5, 2003, entitled “Thrombectomy Catheter Device Having a Self-sealing Hemostasis Valve” now U.S. Pat. No. 7,226,433. The flexible seal 69 is mountingly accommodated by the annular recess 65 of the seal mount 63. The handle body 58 includes a bore 70 partially interrupted by an operation limit slot 78 for slideable accommodation of the high pressure tube mount 60 and for fixed mounting of the seal mount 63 and the strain relief mount 66 therein. Material adjacent to a material devoid cutout section of the handle body 58 provides for inclusion of an arcuate surface 72, an opposed arcuate surface 74, and an offset elongated arcuate section 76 having parallel edges 76a and 76b. The arcuate surface 72, the opposed arcuate surface 74, and the parallel edges 76a and 76b combine to form the operation limit slot 78 which, in part, is used to limit longitudinal and rotational positioning of the high pressure tube mount 60 and, accordingly, the positioning of the flexible high pressure tube 28 and nitinol tube 26 of the jet body 12. The high pressure tube mount 60 can be limitly, slideably and rotationally positioned within the bore 70 of the handle body 58 and is constantly in close proximity and/or contact with each of the arcuate surfaces 72 and 74 and each of the parallel edges 76a and 76b of the operation limit slot 78. Other features cooperate with the operation limit slot 78 to limit the longitudinal and rotational positioning of the high pressure tube mount 60, as later described in detail. The high pressure tube mount 60 includes a longitudinal bore 80, including perpendicular bores extending therefrom, including an adhesive supply bore 82 and a keyway bore 84 communicating with the bore 80. The tubular actuator 62 has a suitably sized longitudinal bore 86 for sliding over the exterior surface of the handle body 58. An adhesive supply bore 88 extends through the wall of the tubular actuator 62. Adhesive application can take place through the adhesive supply bore 88, through the operation limit slot 78, and through the adhesive supply bore 82 to deliver an adhesive to the bore 80 of the high pressure tube mount 60 for fixation of the flexible high pressure tube 28 within the bore 80. A threaded hole 90 extends through the wall of the tubular actuator 62 for accommodation of a flush mounted index screw 92. The index screw 92 is also accommodated by the key bore 84 of the high pressure tube mount 60 to fixingly attach the high pressure tube mount 60 within the tubular actuator 62. As shown in FIG. 5, the index screw 92 is also used to operate within and can limitingly contact the boundaries of the operation limit slot 78 in the handle body 58, whereby longitudinal movement and rotational movement of the high pressure tube mount 60 and the connected tubular actuator 62, as well as the portion of the jet body 12 which is adhesively secured within the high pressure tube mount 60 and that portion of the jet body 12 which is distal to the high pressure tube mount 60, is restricted within a suitable operational range.

FIG. 5 is an assembled view of the components of FIG. 4. Especially illustrated is the relationship of the flexible high pressure tube 28, including the coil 30, to the control handle 16. The portion of the flexible high pressure tube 28 distal to the coil 30 extends through the bore 80 of the high pressure tube mount 60 and is secured therein by an adhesive or by other suitable means, partially through the bore 70 of the handle body 58, through the bore 64 and annular recess 65 of the seal mount 63, through the seal 69, through the bore 67 of the strain relief mount 66, through the bore 68 of the strain relief 20, and through the lumen of the flexible catheter tube 14, as previously described. The flexible high pressure tube 28 is of expandable length and can be positioned along the longitudinal axis of the invention by slidingly positioning the high pressure tube mount 60 within the bore 70 of the handle body 58, the positioning of which is limited by the relationship of the index screw 92 to the operational limit slot 78. The high pressure tube mount 60 is shown with the index screw 92 positioned at the bottom of the operation limit slot 78, i.e., the six o'clock position. The high pressure tube mount 60, which is shown in a mid-longitudinal position with respect to the operation limit slot 78, can be moved proximally or distally along the longitudinal axis of and within the bore 70 of the handle body 58, as well as being rotationally positionable about the longitudinal axis of the handle body 58. During such longitudinal positioning of the high pressure tube mount 60 and the connected flexible high pressure tube 28, the tubular coil 30 is flexed along its length, thereby providing for longitudinal flexibility and elasticity of the flexible high pressure tube 28, whereby the length of the flexible high pressure tube 28 is variable according to the relationship and positioning of the components of the operating handle 16.

FIG. 6 is an isometric view of the guidewire tube exit region 24 where the sheath 22 is shown removed from and distant to the distal end of the flexible coated tube 18. A distally located formed tubular portion 94 of the flexible coated tube 18 includes a truncated and rounded slot 96 of decreasing depth, in a proximal direction, which accommodates the proximal portion of the guidewire tube 57. The truncated and rounded slot 96 is substantially formed in the shape of a nearly full semi-circular arc at the extreme distal end of the distally located formed tubular portion 94. The arc, while the radius remains constant, is decreased progressing proximally from the extreme distal end of the distally located formed tubular portion 94 to provide for angled transitional accommodation of the guidewire tube 57. The proximal end of the guidewire tube 57 is accommodated by the truncated and rounded slot 96 of the flexible coated tube 18 and secured thereto by an adhesive, welding or other such suitable method. Although the guidewire tube 57 is illustrated in a position on top of the flexible high pressure tube 28, such relative positions of each may be in various orientations along the length of the sheath 22.

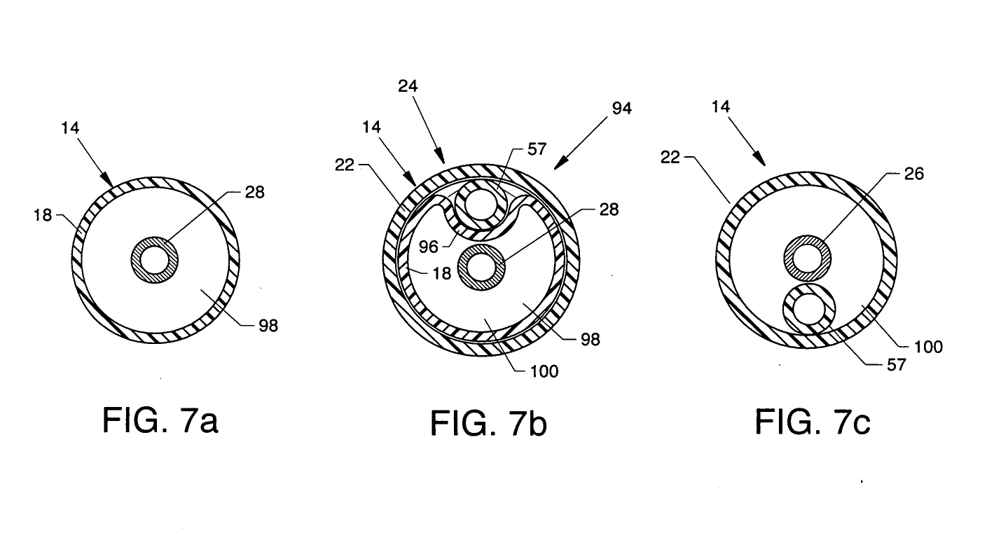

FIGS. 7a, 7b and 7c are cross section views along lines 7a-7a, 7b-7b and 7c-7c of FIG. 1 showing cross section views of the flexible catheter tube 14 and components aligned therein and therealong. Shown in particular in FIGS. 7a, 7b and 7c, respectively, is the lumen 98 of the flexible coated tube 18 and the lumen 100 of the sheath 22, as well as the accommodation of the guidewire tube 57 by the truncated and rounded slot 96 of the formed tubular portion 94 of the flexible coated tube 18.

### Mode of Operation

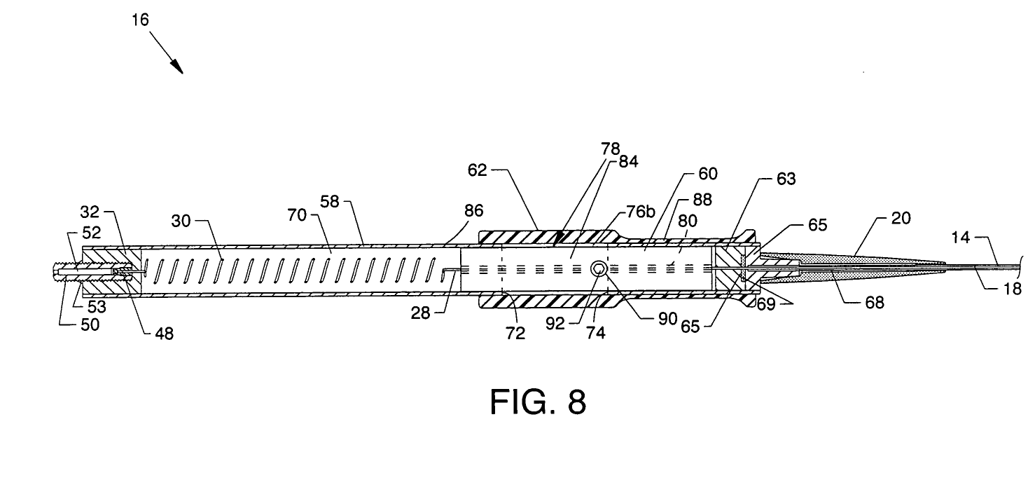

FIGS. 8, 9 and 10 are views showing the various stages of operation of the forwardly directable fluid jet crossing catheter 10.

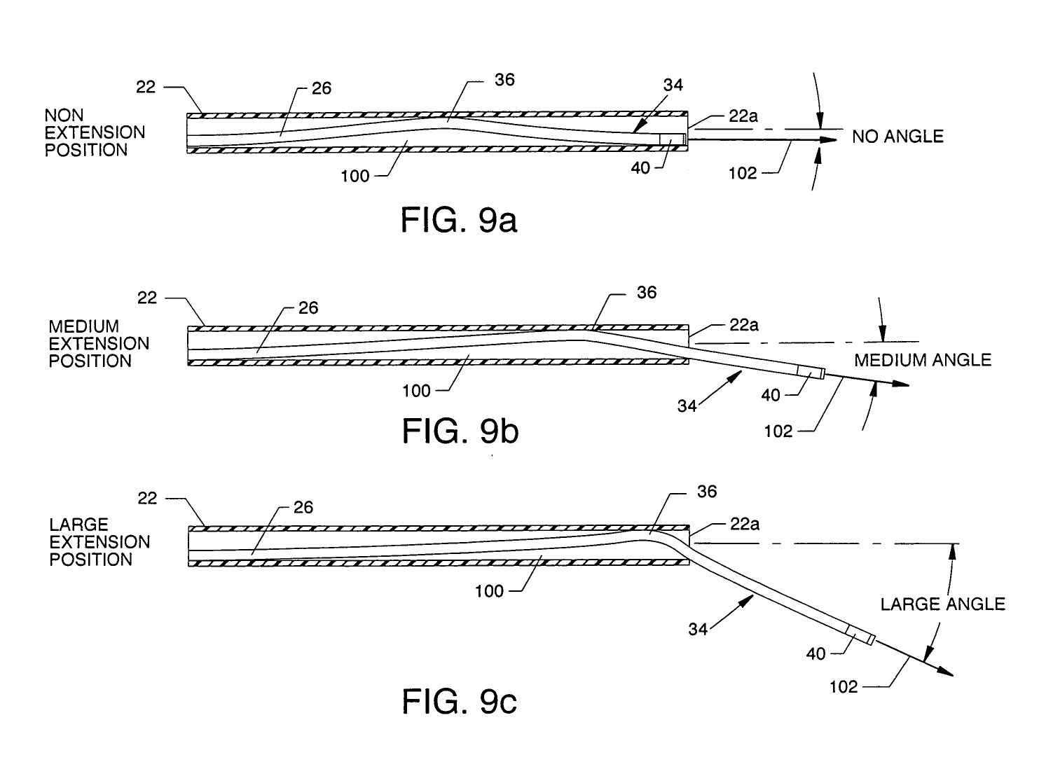

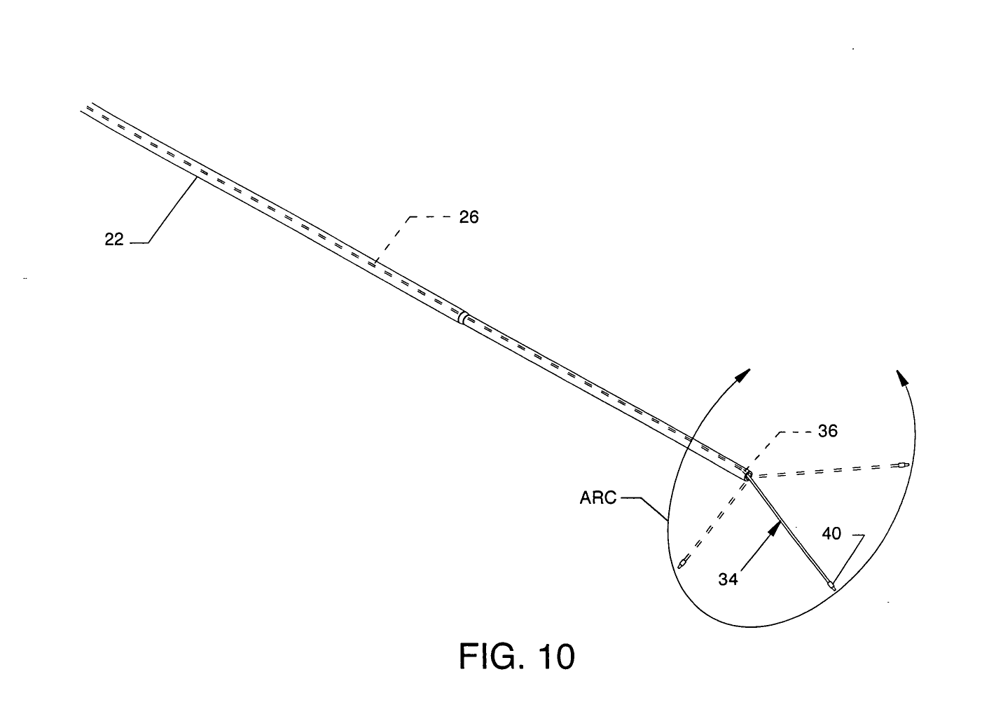

FIG. 8 is a partial cross section view showing the operating handle 16 in relation to the proximally located components of the jet body 28. The high pressure tube mount 60 can be positionally moved by manually grasping and manipulating the proximal end of the handle body 58 and by simultaneous manually grasping and moving the tubular actuator 62 by applying a differential rotational force therebetween and/or by applying differential longitudinally directed force therealong to correspondingly position and orient distally located components of the jet body 12 (FIGS. 2-4). During such manipulation, the high pressure tube mount 60 can be rotated about the longitudinal axis or positioned along the longitudinal axis of the handle body 58 in order to correspondingly cause positioning of the flexible high pressure tube 28, the nitinol tube 26, and thus, the tip 34 of the jet body 12 with respect to the sheath 22, especially at the distal end of the sheath 22 (FIGS. 2 and 3). The high pressure tube mount 60 can be rotated about the longitudinal axis resulting in a plurality of orientations of the tip 34, such as illustrated in FIG. 10. FIG. 8 shows the high pressure tube mount 60 rotated 90° (as viewed from the tip 34 end) in a clockwise direction with reference to the pressure tube mount 60 orientation shown in FIG. 5. In FIG. 8, the high pressure tube mount 60 is also shown positioned fully in the distal direction along the longitudinal axis of the handle body 58, whereby the distally urged position of the tip 34 of the jet body 12 with respect to the sheath 22 is represented in part in FIG. 9c. The index screw 92 is shown impinging the arcuate surface 74 of the operation limit slot 78 to limit the distal longitudinal movement of the high pressure tube mount 60, or conversely the index screw 92 can impinge the arcuate surface 72 of the operational limit slot 78 to limit proximal longitudinal movement of the high pressure tube mount 60. The index screw 92 limits clockwise rotational direction of the high pressure tube mount 60 by impinging the edge 76a of the operation limit slot 78 and is limited in a counterclockwise rotational direction by impinging the edge 76b of the operation limit slot 78. The relationship of the index screw 92 with the operation limit slot 78 is such that positioning of the high pressure tube mount 60 can occur separately, either along or about the longitudinal axis of the handle body 58, or positioning of the high pressure tube mount 60 can occur simultaneously along or about the longitudinal axis of the handle body 58. Separate operation or simultaneous operation can provide for a plurality of orientations of the tip 34.

FIGS. 9a, 9b and 9c illustrate the longitudinal positional and slideable relationships of the tip 34, the bend 36, and a distal portion of the nitinol tube 26 of the jet body 12 to the distal end of the sheath 22 and the lumen 98 thereof during nonextension, medium extension, and large extension positions of the tip 34 near or in a distal direction beyond an interior distal edge 22a of the sheath 22. In FIGS. 9a, 9b and 9c, the distal portion of the nitinol tube 26 generally assumes a nonparallel relationship within the lumen 100 and with the wall of the sheath 22, and correspondingly of the longitudinal axis of the sheath 22, whether extended or not extended beyond the interior distal edge 22a. The tip 34, the bend 36, and the proximally extending portion of the nitinol tube 26 are substantially sprung between and tangentially supported by the interior distal edge 22a, except in FIG. 9a where the tip 34 is supported by the interior wall of the sheath 22, by the inner wall of the sheath 22 at bend 36, and by the proximally extending nitinol tube 26. The interior distal edge 22a of the sheath 22 is of an annular form, a portion of which maintains tangential contact with a tangential portion of the tip 34 when extended, as shown in FIGS. 9b and 9c. In FIGS. 9a, 9b and 9c, the bend 36 firmly and tangentially contacts the wall of the sheath 22 where the angle of the bend 36 is different depending upon the extension length of the tip 34 within the sheath 22 or beyond the interior distal edge 22a of the sheath 22. Another contact being tangential and elongated (not shown) of the proximally extending nitinol tube 26 occurs with and along part of the inner wall of the sheath 22. Accordingly, three contact points or surfaces of various spacings and relationships are provided where the location of such points and the spaced relationship of such points is adjustably variable along the length of the sheath 22 as caused by longitudinal positioning of the high pressure tube mount 60 resulting in a continuous angular range from no angle to medium-to-large angles between the longitudinal axis of the sheath 22 and the tip 34. A fluid jet stream 102 at an appropriate pressure is emitted from the distal end of the tip 34 positioned at desired angles with respect to the longitudinal axis of the sheath 22 for crossing a chronic total occlusion. The distal portion of the guidewire tube 57 is not shown for purposes of brevity and clarity.

FIG. 10 shows the distal portion of the sheath 22 and various rotational positions of the tip 34 rotated therein as the jet body 12 (i.e., the nitinol tube 26) is rotationally positioned by rotational movement of the high pressure tube mount 60 within the bore 70 of the handle body 58. The tip 34 can be rotated about a generous arc thereby providing an ample latitude for suitable orientation of the tip 34, and thus of the fluid jet stream 102 for impingement with CTO material. The entire forwardly directable fluid jet crossing catheter 10 can be rotatingly oriented, as required, to access regions not originally included under influence of the arc. The combination of wide arcuate coverage shown in FIG. 10, with various angles of extension with respect to the longitudinal axis as shown in FIG. 9, provides for a wide expanse of directed fluid jet stream 102 for crossing a chronic total occlusion.

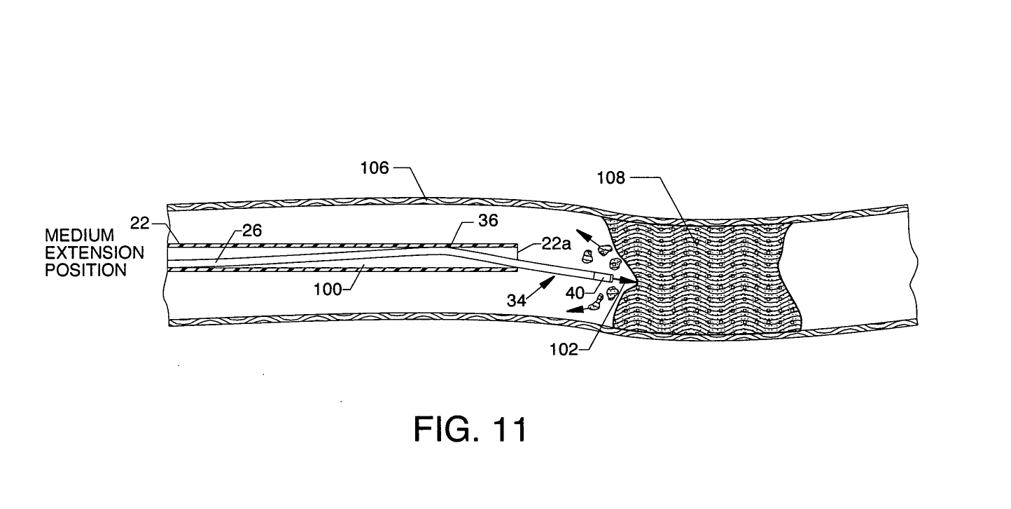

FIG. 11 shows the distal end of the sheath 22 and the tip 34 of the forwardly directable fluid jet crossing catheter 10 aligned in a patient's vessel 106 having a chronic total occlusion 108. For purposes of example and illustration, the tip 34 is positioned at or near the medium extension position, such as shown in FIG. 9b, where the fluid jet stream 102 is distally directed toward the chronic total occlusion 108 in order to provide a path through the chronic total occlusion 108 thereby perfecting a crossing therethrough. During such a procedure, the tip 34 can be longitudinally moved forwardly or rewardly from the position shown for angular variation of the fluid jet stream 102, as well as rotated about the longitudinal axis to radially direct the fluid jet stream 102 by control of the operating handle 16. Subsequent to crossing, a guidewire may be loaded through the guidewire tube exit region 24 and utilized, as may be necessary. Alternatively, subsequent to operation of the tip 34 as described, the guidewire may be utilized as a probe to explore multiple locations in the chronic total occlusion 108 to find a suitable path for crossing the chronic total occlusion 108.

This invention describes a catheter used for purposes of crossing Chronic Total Occlusions (CTO). The forwardly directable fluid jet crossing catheter 10 is compatible with and can be driven by the AngioJet® console described in patent application Ser. No. 11/237,558 filed Sep. 28, 2005, entitled “Thrombectomy Catheter Deployment System”, which is pending. The forwardly directable fluid jet crossing catheter 10 can also be incorporated into use with various support components known in the art. AngioJet® thrombectomy catheters use high velocity jets (>150 m/s) to generate strong secondary flows to liberate, macerate and remove thrombus. The system includes a roller pump to ensure the waste flow is equivalent to the volumetric flow rate of saline pumped into the patient via the high velocity jets known as iso-volumetric flow. In the case of the forwardly directable fluid jet crossing catheter 10, a single mid-range velocity jet (10-80 m/s) is directed forward to seek a path through a chronic total occlusion. The forwardly directable fluid jet crossing catheter 10 may not necessarily have a waste flow that comes out of the patient, so it is not necessarily an iso-volumetric catheter. The directable tip can be useful in assisting the catheter across a CTO since there is no wire across the lesion where at times the CTO can be located in branches of an artery. The directability of the tip 34 is a fundamental element for assisting the physician to cross a lesion. This dynamic directability for the forwardly directable fluid jet crossing catheter 10 is viewed as an advantage over prior art devices. The moderate speed jet emitted from the tip 34 will naturally find a dissection plane in the fibrous material of the CTO. Rather than burning through the toughest part of the CTO because that is where the wire tip happened to end up, the moderate speed jet emitted from the tip 34 will find natural dissection planes through the fibrous material, thus reducing the time to accomplish the treatment. The compatibility of the forwardly directable fluid jet crossing catheter 10 with 0.014 inch guidewires means that a physician could use the forwardly directable fluid jet crossing catheter 10 in combination with other CTO devices. For example, one combination would be the use of a forwardly directable fluid jet crossing catheter 10 used with a very stiff tip guidewire. The very stiff tip guidewire could be used to help breach the fibrous cap and the forwardly directable fluid jet crossing catheter 10 could be used to help support the guidewire and to generate a channel once the cap had been breached. A different combination would be the use of a forwardly directable fluid jet crossing catheter 10 with the previously referenced system, in which case, the RF wire could help breach the fibrous cap and the system could be used to visualize and safety check the path while the forwardly directable fluid jet crossing catheter 10 could expeditiously open the channel at or beyond the cap.

The typical mode of operation for crossing a coronary CTO is a planned procedure which is typically not an emergency situation. In general, a patient with a known CTO means that there was a previous failed attempt to cross a total occlusion with a guidewire where, as a result, a separate intervention may be planned at a later date to cross the CTO in order to provide the patient with a greater flow reserve. Peripheral procedures may involve an extreme difficulty in positioning a guidewire at a distal location. It may be common for the interventionalist to have a set of tools available to assist in crossing these difficult-to-cross occlusions. Some physicians may commonly rely on a laser as an adjunct tool, while others may have a set of guidewires used for negotiating the occlusions. In either case, the mode of operation would be similar. The physician would determine that a particular occlusion needed an adjunct tool to help crossing; in this case, the forwardly directable fluid jet crossing catheter 10, the operation of which can be supported by an AngioJet® console, often referred to as the AngioJet® Ultra System, or in the alternative, can be supported by combinations of other peripheral components. The forwardly directable fluid jet crossing catheter 10 can be combined with a pump in a sterile package using a sterile technique. The pump would be loaded into the AngioJet® console and a supply of heparinzed saline would be connected to the pump via a common bag spike and primed by stepping on a foot switch. The forwardly directable fluid jet crossing catheter 10 would be advanced to the treatment site by riding over the wire. The tip 34 of the forwardly directable fluid jet crossing catheter 10 would be directed at the occlusion and the foot pedal depressed, thus providing a fluid jet stream of saline that would find the natural dissection plane through the CTO, whereby a guidewire would be advanced. Then, the forwardly directable fluid jet crossing catheter 10 would be advanced and the process repeated until the occlusion was crossed. Once the occlusion was crossed, the intervention to treat the occlusion with either atherectomy or stenting could proceed.

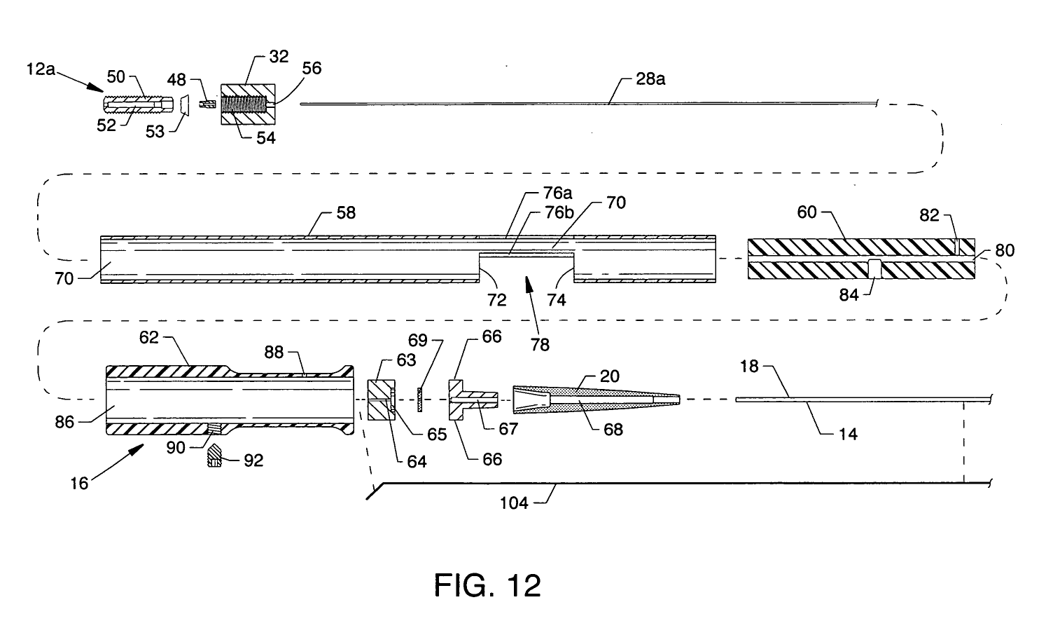

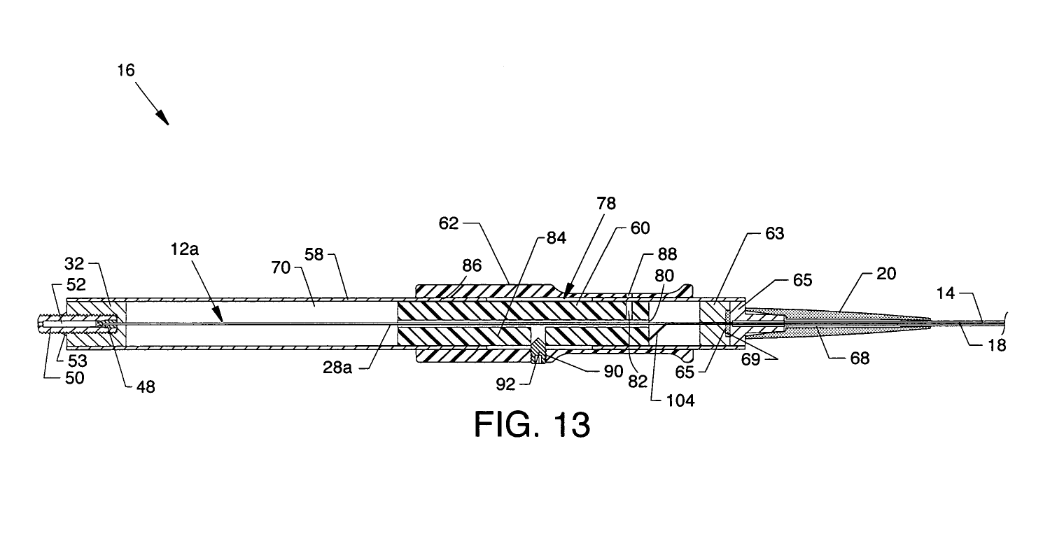

FIG. 12 is an exploded view similar to FIG. 4 showing the proximal portion of an alternative embodiment of a jet body 12a for use in lieu of the jet body 12 shown in FIG. 2. FIG. 13 is similar to FIG. 5 showing an assembled view of the proximal portion of an alternative embodiment of a jet body 12a used in lieu of the jet body 12. A control wire 104 is included in the jet body 12a for use in directing the tip 34 at the distal end of the jet body 12a. In the illustrations it is noted that the coil 30 is not utilized and, as such, the flexible high pressure tube 28 is substantially straight and is referred to as the flexible high pressure tube 28a as the need for an expandable flexible high pressure tube length is not required. In this arrangement, the high pressure tube mount 60 is not attached to the flexible high pressure tube 28a by the use of adhesive in the bore 80, such as in the jet body 12 of FIG. 2. The high pressure tube mount 60 can traverse along a portion of the bore 86 of the handle body 58 and along a corresponding portion of the flexible high pressure tube 28a where the bore 80 of the high pressure tube mount 60 passingly accommodates a portion of the flexible high pressure tube 28a. The proximal end of the control wire 104 is attached to the distal end of the high pressure tube mount 60 and the control wire 104 is movable forwardly and rewardly by said tube mount 60. The control wire 104 sharingly continues and extends through the bore 64 of the seal mount 63, through the seal 69, through the bore 67 of the strain relief mount 66, and thence along the lumen 98 of the flexible coated tube 18, along the guidewire tube exit region 24, through the lumen 100 of the sheath 22 to suitably attach to the nitinol tube 26 at a location slightly distal to the bend 36 near the tip 34.

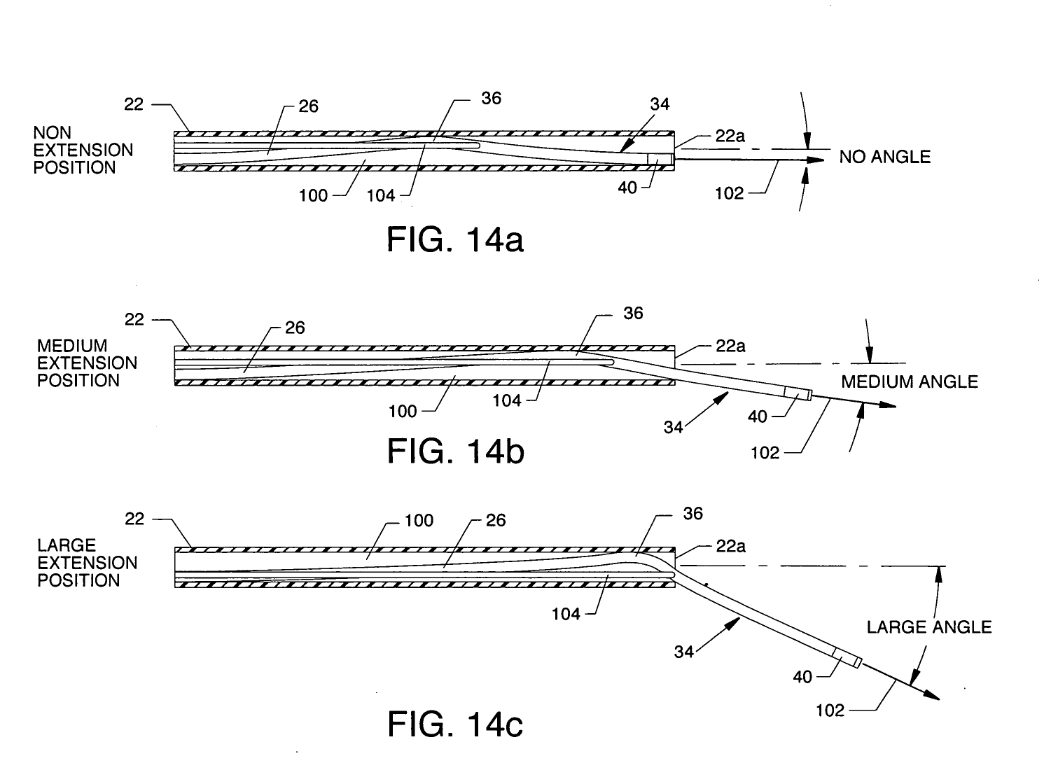

FIGS. 14a, 14b and 14c are similar to FIGS. 9a, 9b and 9c showing the attachment of the distal end of the control wire 104 to the nitinol tube 26 at a location just distal of the bend 36. As in FIGS. 9a, 9b and 9c, three contact points of various spacings and relationships are provided where the location of such points and the spaced relationships of such points is adjustably variable along the length of the sheath 22 as caused by the longitudinal positioning of the high pressure tube mount 60 resulting in a continuous angular range from no angle to medium-to-large angles between the longitudinal axis of the sheath 22 and the tip 34.

A fluid jet stream 102 at an appropriate pressure is emitted from the distal end of the tip 34 positioned at a desired angle with respect to the longitudinal axis of the sheath 22 for crossing a chronic total occlusion. The entire forwardly directable fluid jet crossing catheter 10 using the jet body 12a can be rotatingly oriented as required in a full arc for full arcuate fluid jet stream orientation. The distal portion of the guidewire tube 57 is not shown for purposes of brevity and clarity.

Operation of and teaching of the forwardly directable fluid jet crossing catheter 10 using the jet body 12a closely parallels that of the operation and teachings of the forwardly directable fluid jet crossing catheter 10 using the jet body 12.

Various modifications can be made to the present invention without departing from the apparent scope thereof.

### PARTS LIST

10 forwardly directable fluid jet crossing catheter
12 jet body
12a jet body
14 flexible catheter tube
16 operating handle
18 flexible coated tube
20 strain relief
22 sheath
22a interior distal edge
24 guidewire tube exit region
26 nitinol tube
28 flexible high pressure tube
28a flexible high pressure tube
30 coil
32 high pressure tube adapter
34 tip
36 bend
40 radiopaque marker band
42 radiopaque marker band
44 radiopaque marker band
46 radiopaque marker band
48 ferrule
50 high pressure connector
52 bore
53 adhesive
54 threaded hole
56 bore
57 guidewire tube
58 handle body
60 high pressure tube mount
62 tubular actuator
63 seal mount
64 bore
65 annular recess
66 strain relief mount
67 bore
68 configured bore
69 seal
70 bore (of 58)
72 arcuate surface
74 arcuate surface
76 elongated acruate section
76a-b edges
78 operation limit slot
80 bore
82 adhesive supply bore
84 key way bore
86 bore
88 adhesive supply bore
90 threaded hole
92 index screw
94 formed tubular portion
96 truncated and rounded slot
98 lumen (of 18)
100 lumen (of 22)
102 fluid jet stream
104 control wire
106 vessel
108 chronic total occlusion

1. A fluid jet crossing catheter comprising an elongated flexible catheter tube attached to an operating handle, said catheter tube including a flexible, elongated tubular member having a proximal end and a distal end, said operating handle having an elongated tubular body with a proximal end and a distal end, said proximal end of said flexible, elongated tubular member being connected through the distal end of said elongated tubular body, a flexible, high pressure, elongated tube extending from within said elongated tubular body and through said flexible, elongated, tubular member, said flexible, high pressure, elongated tube having a movable tip which can be extended beyond said distal end of said flexible, elongated tubular member, said flexible, high pressure, elongated tube having a bent portion within said flexible, elongated tubular member, and said movable tip being controlled by a mechanism associated with said operating handle.

2. The fluid jet crossing catheter of claim 1, wherein said flexible, elongated tubular member has a first elongated tubular section and a second elongated tubular section, said first tubular section having a proximal end and a distal end, said second tubular section having a proximal end and a distal end, said proximal end of said first tubular section being attached to said distal end of said elongated tubular body, said distal end of said first elongated tubular section being attached to said proximal end of said second elongated tubular section.

3. The fluid jet crossing catheter of claim 2, wherein said bent portion of said flexible high pressure tube is located within said second elongated tubular section.

4. The fluid jet crossing catheter of claim 1, wherein said mechanism of said operating handle for controlling said movable tip includes a slideable and rotatable tubular actuator external to said elongated tubular body and a slideable and rotatable tubular mount internal to said elongated tubular body, said flexible, high pressure, elongated tube being fixedly attached to said internal tubular mount and said external tubular actuator and said internal tubular mount being in operative mechanical conjunction with each other to move said movable tip externally of said distal end of said second elongated tubular section in either a horizontally forward or rearward direction or in a rotational direction.

5. The fluid jet crossing catheter of claim 1, wherein said operating handle has a bored adapter sealed to the proximal end of said elongated tubular body and a tubular connector sealed to said bored adapter for the input of a pressurized fluid into said flexible, high pressure, elongated tube within said elongated tubular body.

6. (canceled)

7. (canceled)

8. The fluid jet crossing catheter of claim 1, wherein said flexible, high pressure, elongated tube has a third elongated tubular section and a fourth elongated tubular section, said third and fourth elongated tubular sections being connected to each other in tandem.

9. The fluid jet crossing catheter of claim 8, wherein said bent portion is within said second elongated tubular section.

10. The fluid jet crossing catheter of claim 9, wherein said third elongated tubular section is joined to said fourth elongated tubular section within said second elongated tubular section on the proximal side of said bent portion.

11. The fluid jet crossing catheter of claim 8, wherein said third tubular section is made from stainless steel and said fourth tubular section is made from nitinol.

12. The fluid jet crossing catheter of claim 11, wherein said tip, at said distal end of said fourth elongated tubular section, is annealed.

13. The fluid jet crossing catheter of claim 9, wherein portions of said fourth tubular section adjacent said bent portion are super-elastic as well as said bent portion.

14. The fluid jet crossing catheter of claim 5, wherein said flexible, elongated, high pressure tube includes a flexible tubular coil extending between said tubular connector and said flexible, elongated, high pressure tube, said flexible tubular coil forming an integral part of said flexible, elongated, high pressure tube.

15. The fluid jet crossing catheter of claim 2, wherein said distal end of said first elongated tubular section is attached to said proximal end of said second elongated tubular section by insertion thereof a short distance into said proximal end of said second elongated tubular section and is secured thereto by an adhesive, electric welding or some other suitable means.

16. The fluid jet crossing catheter of claim 2, wherein said first elongated tubular section has a curved indenture in the periphery thereof near its distal end for insertion of a guidewire tube which extends internally from said proximal end of said second elongated tubular section to said distal end of said second elongated tubular section.

17. The fluid jet crossing catheter of claim 1, wherein there is a radiopaque marker band on said movable tip and at least one radiopaque marker band on said flexible, elongated tubular member.

18. The fluid jet crossing catheter of claim 1, wherein said operating handle has a flexible strain relief member sealingly attached to the distal end of said elongated tubular body, said flexible strain relief member having a bore therethrough and a conically shaped outer surface wherein said flexible, elongated, tubular member extends through said bore.

19. A fluid jet crossing catheter comprising an elongated flexible catheter tube attached to an operating handle, said catheter tube including a flexible, elongated, tubular member having a proximal end and a distal end, said operating handle having an elongated tubular body with a proximal end and a distal end, said elongated tubular body having a bored adapter sealed to the proximal end of said elongated tubular body and a tubular connector sealed to said bored adapter for the input of a pressurized fluid, a flexible, high pressure, elongated tube being fixed to said bored adapter and in fluid communication with said tubular connector, said flexible, high pressure, elongated tube extending from said bored adapter, through said elongated tubular body, through said distal end of said elongated tubular body and through said elongated tubular member, said flexible, high pressure, elongated tube having a movable tip at said distal end of said flexible, elongated tubular member which movable tip can be extended beyond said distal end of said flexible, elongated tubular member, said flexible, high pressure, elongated tube having a bent portion within said flexible, elongated tubular member, and said movable tip being controlled by a mechanism associated with said operating handle.

20. The fluid jet crossing catheter of claim 19, wherein said mechanism includes a control wire having a proximal end and a distal end, said proximal end of said control wire being fixedly attached internally of said elongated tubular body and said distal end of said control wire being fixedly attached to and adjacent said bent portion within said flexible, elongated tubular member.

21. The fluid jet crossing catheter of claim 20, wherein said mechanism of said operating handle for controlling said movable tip further includes a slideable and rotatable tubular actuator external to said elongated tubular body and a slideable and rotatable tubular mount internal to said elongated tubular body, said slideable and rotatable tubular mount having a proximal end and a distal end, said proximal end of said control wire having an off center portion fixedly attached to said distal end of said slideable and rotatable tubular mount, said distal end of said control wire being fixedly attached to said flexible, high pressure, elongated tube adjacent the downstream side of said bent portion of said flexible, high pressure, elongated tube, said tubular actuator and said tubular mount being in operative mechanical conjunction with each other to move said control wire in a horizontal or rotational direction whereby said movable tip is moved externally of said distal end of said flexible, elongated, tubular member in either a horizontally forward or rearward direction or in a rotational direction.

22. The fluid jet crossing catheter of claim 21, wherein said flexible, elongated tubular member has a first elongated tubular section and a second elongated tubular section, said first elongated tubular section having a proximal end and a distal end, said second elongated tubular section having a proximal end and a distal end, said proximal end of said first section being attached to said distal end of said elongated tubular body, said distal end of said first section being attached to said proximal end of said second section.

23. The fluid jet crossing catheter of claim 22, wherein said bent portion of said flexible, high pressure, elongated tube is located within said second tubular section.

24. (canceled)

25. (canceled)

26. The fluid jet crossing catheter of claim 19, wherein said flexible, high pressure, elongated tube has a third elongated tubular section and a fourth elongated tubular section, said third and fourth elongated tubular sections being connected to each other.

27. The fluid jet crossing catheter of claim 26, wherein said bent portion is within said second elongated tubular section.

28. The fluid jet crossing catheter of claim 27, wherein said third elongated tubular section is joined to said fourth elongated tubular section within said second elongated tubular section on the proximal side of said bent portion.

29. The fluid jet crossing catheter of claim 28, wherein said third tubular section is made from stainless steel and said fourth tubular section is made from nitinol.

30. The fluid jet crossing catheter of claim 29, wherein said movable tip, at said distal end of said second section, is annealed.

31. The fluid jet crossing catheter of claim 30, wherein a portions of said fourth tubular section proximal to said bent portion are super-elastic as well as said bent portion.

32. The fluid jet crossing catheter of claim 19, wherein said distal end of said first elongated tubular section is attached to said proximal end of said second elongated tubular section by insertion thereof a short distance into said proximal end of said second elongated tubular section and is secured thereto by an adhesive, electric welding or some other suitable means.

33. The fluid jet crossing catheter of claim 32, wherein said first tubular section has a curved indenture in the periphery thereof near its distal end for insertion of a guidewire tube which extends internally from said proximal end of said second elongated tubular section to said distal end of said second elongated tubular section.

34. The fluid jet crossing catheter of claim 19, wherein there is a radiopaque marker band on said movable tip and at least one radiopaque marker band on said first elongated tubular section.

35. The fluid jet crossing catheter of claim 19, wherein said elongated tubular body has a flexible strain relief member sealingly attached to the distal end thereof, said flexible strain relief member having a bore therethrough and a conically shaped outer surface wherein said flexible, elongated, tubular member extends through said bore.

36. The fluid jet crossing catheter of claim 21, wherein there is a threaded hole located approximately midway between the ends of said tubular actuator for insertion of an index screw cooperating with a key bore in said tubular mount to fixedly prevent the movement thereof or cooperating with a limit slot in said elongated tubular body to limit the rotational and longitudinal movement of said internal tubular mount.

37. The fluid jet crossing catheter of claim 4, wherein there is a threaded hole located approximately midway between the ends of said tubular actuator for insertion of an index screw cooperating with a key bore in said tubular mount to fixedly prevent the movement thereof or cooperating with a limit slot in said elongated tubular body to limit the rotational and longitudinal movement of said internal tubular mount.
